(19) United States
(12) Patent Application Publication
(10) Pub. No.: US 2008/0229512 A1
(43) Pub. Date: Sep. 25, 2008

(54) Conditioning Hair Lightener System, Compositions, Method and Kit Therefor

(76) Inventors: Ali N. Syed, Inverness, IL (US); Narjis A. Askar, Naperville, IL (US); Tomas N. Ventura Jr, Lisle, IL (US); Khadijeh Saad, Bolingbrook, IL (US)

Correspondence Address: Olson & Cepuritis, LTD., 20 NORTH WACKER DRIVE, 36TH FLOOR, CHICAGO, IL, 60606, US

(21) Appl. No.: 10/586,100

(22) PCT Filed: Jan. 28, 2005
(86) PCT No.: PCT/US05/03462

§ 371 Date: Jul. 14, 2006

Related U.S. Application Data
(60) Provisional application No. 60/540,176, filed on Jan. 29, 2004.

(51) Int. Cl. A61K 8/18; A61Q 5/08

(52) U.S. Cl. 8/111; 424/62

(57) ABSTRACT

A conditioning hair lightener system, compositions, method, and kit therefor, is disclosed which ameliorates the deleterious effects of chemical oxidative hair lightening on the strength and subjective properties of hair. The conditioning hair lightener system comprises a conditioning hair lightener emulsion having a pH of at least about 8 prepared from at least two separate components, (A) and (B). Component (A) preferably is a substantially anhydrous, substantially free-flowing composition comprising an effective hair conditioning amount of a water-dispersible, self-emulsifying, fatty acid-derived conditioner, an effective hair lightening amount of a peroxy salt compound, optionally an effective hair protective amount of hair protectant, deswelling agent, and optionally, a water-soluble cosmetic adjuvant. Component (B) preferably is an aqueous medium containing hydrogen peroxide or hydrogen peroxide source. Components (A) and (B) are maintained separate until substantially immediately before use, and are mixed together to provide a conditioning hair lightener emulsion.

### CROSS-REFERENCE TO RELATED APPLICATION

This application claims the priority of U.S. Provisional Application for Patent Ser. No. 60/540,176 filed on Jan. 29, 2004, which is incorporated herein by reference.

### TECHNICAL FIELD OF THE INVENTION

This invention relates to the modification of the natural color of hair, and in particular, to lightening of hair color while ameliorating logs of hair strength.

### BACKGROUND OF THE INVENTION

People like to change the color of their hair if it is not satisfactory, or simply as a fashion whim, by either chemically dyeing the hair to a different color or by lightening its natural color. In particular, lightening of human hair has been widely practiced since Roman times and is now a well known cosmetic process.

Chemical lightening or bleaching of the natural hair pigment is recognized as an oxidative process and the most satisfactory oxidizing agent is an aqueous solution of hydrogen peroxide (or source thereof). In practice, the oxidizing action of hydrogen peroxide is activated by increasing the pH to an alkaline pH, typically with ammonium hydroxide (or source thereof), and can be accelerated by increasing the alkalinity to a pH value in the range of about 8 to about 11.5, and including peroxy salts, such as persulfates, percarbonates, perborates, and the like, as “boosters” or “accelerators”.

The depth of color on hair is conventionally described in terms of levels classified by a range of 12 shades, with level 1 being the darkest (i.e., black) and level 12 being the lightest (i.e., ultra light blonde). The shade levels assigned to this numbering system are described by Brown, K. C., et al., in the SCC Monograph, Permanent Hair Dyes, p. 22, published by the Society of Cosmetic Chemists (1996).

The practice of chemically lightening the natural color of hair to varying shade levels typically involves applying an oxidizing agent, generally liquid hydrogen peroxide (or hydrogen peroxide source) to the hair, and leaving it in contact with the hair for a sufficient period of time to effectively achieve a desired lighter hair shade. The degree of lightening can be varied by: a) gradually lightening or highlighting the hair with either acidic hydrogen peroxide or brief multiple treatments with alkaline hydrogen peroxide, or b) decoloring the hair several levels by contacting the hair for prolonged periods of up to about one hour or longer with alkaline, generally ammoniacal, hydrogen peroxide-containing compositions. For example, by bleaching the hair more than 3 to 4 levels, a person with dark hair can achieve a blonde shade. A change of 6 to 7 levels in the color of dark hair is usually the maximum practical reduction achieved with strong alkaline bleaches, i.e., having a relatively high alkaline pH value above about 9.

Most strongly alkaline bleaching is typically accomplished with ammoniacal, high volume hydrogen peroxide and peroxy salt boosters, such as sodium persulfate, potassium persulfate, ammonium persulfate and mixtures thereof. Formulations of this type commonly have at least two and usually all three of the persulfate salts, with ammonium persulfate providing an ammonia source. Such strongly alkaline bleaches, however, are known to damage the hair extensively by swelling the hair, thereby structurally weakening the tensile properties of the fibers resulting in breakage, and negatively affecting the aesthetic subjective properties resulting in a delustered appearance and straw-like feel, especially as the contact time on the hair is prolonged.

There is an ongoing need and desire, therefore, for compositions and processes for achieving hair lightening at a relatively high alkaline pH with minimal hair damage. The present invention provides a conditioning hair lightener system, compositions, method, and kit therefor, which ameliorates the deleterious action of chemical oxidative lightening on the hair.

### SUMMARY OF THE INVENTION

A conditioning hair lightener system is disclosed which ameliorates the deleterious action of chemical oxidative lightening on the strength and subjective properties of human hair. The conditioning hair lighter system comprises at least two separate components, (A) and (B), which are admixed with one another substantially immediately before use to provide a conditioning hair lightener.

Component (A) is a substantially anhydrous, substantially free-flowing composition comprising an effective hair conditioning amount of a water-dispersible, self-emulsifying, fatty acid-derived conditioner, an effective hair-lightening amount of at least one peroxy salt, optionally an effective hair protective amount of hair protectant, deswelling agent, and optionally a water-soluble cosmetic adjuvant. Component (B) is an aqueous medium preferably containing hydrogen peroxide or a hydrogen peroxide source. Component (A) substantially immediately forms an emulsion when mixed with Component (B). Component (A) preferably includes sufficient alkaline ingredients to provide a conditioning hair lightener emulsion having a pH of at least about 8.

The self-emulsifying, fatty acid-derived conditioner preferably comprises a fatty ester, an ethoxylated glyceride, a fatty alcohol, a fatty ether, and any combination thereof, and any formulation thereof optionally containing one or more hydrophilic surfactant, such as an anionic surfactant, a nonionic surfactant, and the like. A particularly preferred self-emulsifying, fatty acid-derived conditioner for use in Component (A) comprises a water-dispersible, self-emulsifying combination of esters of C₆-C₂₂ fatty acids. The combination of esters preferably comprises at least one polyhydric ester selected from the group consisting of: (a) a C₃-C₄ polyol ester of a C₆-C₂₂ fatty acid, (b) a glyceryl ester of a C₆-C₂₂ fatty acid and at least one acid selected from the group consisting of citric acid, lactic acid and succinic acid, and (c) a polyethoxylated C₁₂-C₁₈ acylated sorbitol ester. The peroxy salt is preferably selected from the group consisting of an alkali metal persulfate, ammonium persulfate, and mixtures thereof.

For lightening hair under relatively high alkaline conditions at a pH of at least about 9, the hair lightener emulsion preferably contains a total amount of self-emulsifying, fatty acid-derived conditioner in the range of about 0.2 to about 6% based on the weight of the emulsion. Preferably either one of Component (A) or Component (B), more preferably both, contains a hair protectant, deswelling agent.

A conditioning hair lightener system of this invention can also include at least one of a post-lightener acidic hair conditioner having a pH of not more than about 5, a post-lightener cationic hair conditioner, a post-lightener shampoo preferably having a pH in the range of about 4 to about 6. Preferably the conditioning hair lightener system is provided in a kit form, optionally including one or more hair lightening implements.

A preferred method of lightening hair comprises preparing a conditioning hair lightener emulsion by admixing Component (A) and Component (B), as described herein, substantially immediately before use, contacting the hair with sufficient conditioning hair lightener emulsion for a time period sufficient to achieve the level of lightening desired, and removing the hair lightener emulsion from the hair, as by water rinsing. In a particularly preferred method aspect, the lightened hair is contacted with a post-lightener acidic conditioner prior to, concurrently with, or after removal of the hair lightener emulsion from the lightened hair.

Surprisingly, a conditioning hair lightener system of this invention beneficially ameliorates one or more deleterious effect on the tensile and tactile properties of hair oxidatively lightened under relatively high alkaline conditions.

### DETAILED DESCRIPTION OF PREFERRED EMBODIMENTS

The term “conditioning” as applied to a hair lightener emulsion includes the amelioration of at least one deleterious undesirable effect conventionally associated with the action of alkaline and oxidative products on the measurable integrity of the hair fibers, i.e., a lessening of the tensile strength, loss in wet elasticity, an increase in alkali solubility, a decrease in ease of combing, as well as changes in the subjective properties, i.e., loss of a smooth tactile feel and diminished lustrous appearance.

The terms “substantially anhydrous,” and “dry weight” are used interchangeably herein to mean that an amount of water, present either as free water, water of hydration, or water of reaction, is not more than about fifteen weight percent, preferably not more than about ten weight percent, more preferably not more than about five weight percent, as applied to an ingredient, and is not more than about ten weight percent, preferably not more than about five weight percent, more preferably not more than about two weight percent, as applied to a formulated Component (A) composition.

The preferred hair lightener embodiments are described primarily for lightening the color of scalp hair but are not limited thereto. The term “hair” is intended to also encompass hair on the body, including the face, trunk and limbs.

The term “polyhydric” includes compounds having at least 3 carbon atoms and at least 2 hydroxyl groups.

Cosmetic ingredients, additives, products or materials, and optional cosmetic adjuvants, that can be employed in the hair lightener compositions and system discussed herein are referred to by their commonly used chemical names or by the international nomenclature commonly referred to as INCI name given them in any edition of the International Cosmetic Ingredient Dictionary and Handbook, (hereafter INCI Dictionary), such as found in Volumes 1-3, of the Seventh Edition (1997) or Eighth Edition (2000) or Ninth Edition (2002), all published by the Cosmetic, Toiletry, and Fragrance Association, Washington D.C. Numerous commercial suppliers of materials listed by INCI name, trade name, or both, can be found in any edition of the INCI Dictionary and in numerous commercial trade publications, including but not limited to, the 2001 Cosmetic Bench Reference, edition of COSMETICS & TOILETRIES®, 115 (13), published by Allured Publishing Corporation, Carol Stream, Ill. (2001), and the 2001 McCutcheon 's Directories, Volume 1: Emulsifiers & Detergents and Volume 2: Functional Materials, published by McCutcheon's Division, The Manufacturing Confectioner Publishing Co., Glen Rock, N.J. (2001); the relevant disclosures of the INCI Dictionary and each of the foregoing publications being incorporated herein by reference.

The term “cosmetic adjuvant” includes cosmetically useful product finishing and promotional additives, well known and conventionally used in the cosmetic arts to maintain the free flow properties of a substantially anhydrous composition, the physical stability of a composition during storage (shelf life), and the visible aesthetic appearance of a composition during storage and during the use of the composition. Cosmetic adjuvants that maintain the stability of products typically include a metal-ion chelating agent, an antioxidizing agent, a preservative, an emulsifying agent, a perfume solubilizer, and the like, but are not limited thereto. Cosmetic adjuvants, sometimes called promotional ingredients, aid in enhancing the aesthetics and marketing appeal of the product and include, without limitation, a product colorant, a fragrance, and the like.

The term “self-emulsifying, fatty acid-derived conditioner” as used herein, and in the appended claims, refers to a fatty ester, an ethoxylated glyceride, a fatty alcohol, a fatty ether, and combinations thereof, and formulations thereof optionally containing one or more hydrophilic surfactant, such as an anionic surfactant, a nonionic surfactant, and the like, wherein the conditioner is water dispersible and substantially immediately forms an emulsion in an aqueous medium at an ambient temperature preferably in the range of about 10 to about 35° C.

Fatty esters include, without limitation thereto, mono-, di-, and tri-, C₃-C₄ polyol esters of C₆-C₂₂ fatty acids and mixtures thereof, such as caprylic/capric triglyceride, caprylic/capric/lauric triglyceride, caprylic/capric/myristic/stearic triglyceride, vegetable oils, fats, glyceryl distearate, glyceryl stearate, glyceryl caprylate, glyceryl caprate/caprylate, propylene glycol stearate, butylene glycol stearate, and the like; a glyceryl ester of a C₆-C₂₂ fatty acid and at least one acid selected from the group consisting of citric acid, lactic acid and succinic acid (e.g., glyceryl cocoate/citrate/lactate), and the like; fatty esters of carbohydrates and reduced carbohydrates, such as polyethoxylated C₁₂-C₁₈ acylated sorbitol ester (e.g., PEG-40 sorbitan peroleate), C₁₂-C₁₈ acylated sorbitol esters, sucrose esters, glucose esters, and the like; fatty alcohol esters of fatty acids, such as cetyl palmitate, cetyl myristate, and the like, and esters of C₂-C₁₀ aliphatic alcohols and fatty acids, such as 2-ethylhexyl pelargonate (octyl perlargonate), isopropyl myristate, and the like.

Ethoxylated glycerides include, without limitation thereto, ethoxylated hydroxy-substituted triglycerides, ethoxylated diglycerides, and ethoxylated monoglycerides, such as polyethoxylated castor oil, polyethoxylated glyceryl stearate, and the like.

Preferred fatty alcohols include, without limitation thereto, cetyl alcohol, stearyl alcohol, lauryl alcohol, oleyl alcohol, and the like.

Fatty ethers include, without limitation thereto, polyethoxylated fatty alcohols, such as polyethoxylated cetyl alcohol, polyethoxylated stearyl alcohol, and mixtures thereof, (polyethoxylated cetearyl alcohol), and the like; fatty alcohol glycosides, such as cetearyl glucoside, and the like.

Particularly preferred are fatty esters derived from vegetable oils, such as coconut oil, palm kernel oil, castor oil, babassu oil, soybean oil, peanut oil, corn oil, sunflower seed oil, grape seed oil, safflower oil, meadowfoam oil, olive oil, palm oil, wheat germ oil, avocado oil, almond oil, and the like, without being limited thereto, with coconut oil and palm kernel oil being preferred.

A particularly preferred self-emulsifying, fatty acid-derived conditioner for use in Component (A) comprises a water-dispersible, self-emulsifying combination of esters of C₆-C₂₂ fatty acids containing at least one polyhydric ester selected from the group consisting of: (a) a C₃-C₄ polyol ester of a C₆-C₂₂ fatty acid, (b) a glyceryl ester of a C₆-C₂₂ fatty acid and at least one acid selected from the group consisting of citric acid, lactic acid and succinic acid, and (c) a polyethoxylated C₁₂-C₁₈ acylated sorbitol ester. Particularly preferred is a combination of caprylic/capric triglyceride, glyceryl cocoate/citrate/lactate (INCI name for a product sold under the tradename INWITOR® 380), and PEG-40 sorbitan peroleate.

Suitable water dispersible, self-emulsifying, fatty acid-derived conditioners may also include commercially available formulations having the INCI names, glyceryl stearate SE, a self-emulsifying grade of glyceryl stearate that contains some sodium and/or potassium stearate (such as LIPO® GMS 470); cetearyl alcohol(and)sodium cetearyl sulfate (such as LANETTE® N); cetearyl alcohol(and)sodium lauryl sulfate(and)sodium cetearyl sulfate (such as LANETTE® SX); cetearyl alcohol(and)sodium lauryl sulfate (such as LANETTE® W); cetearyl alcohol(and)PEG-40 castor oil(and)sodium cetearyl sulfate (such as EMULGADE® F), cetearyl alcohol(and)PEG-40 castor oil (such as EMULGADE® SPEC); cetearyl glucoside(and)cetearyl alcohol (such as EMULGADE® PL68/50); cetearyl alcohol(and)ceteareth-20 (such as EMULGADE® 100NI); glyceryl stearate(and)ceteareth-20(and)ceteareth-10 (and)cetearyl alcohol(and)cetyl palmitate (such as EMULGADE® SE); and the like. A preferred combination is glyceryl stearate SE and caprylic/capric triglyceride; glyceryl stearate SE and glyceryl cocoate/citrate/lactate; and glyceryl stearate SE and PEG-40 sorbitan peroleate.

In a preferred embodiment of Component (A), the amount of self-emulsifying, fatty acid-derived conditioner is in the range of about 0.1 to about 10 weight percent, preferably in the range of about 0.5 to about 8 weight percent, more preferably in the range of about 0.75 to about 5 weight percent, of the total weight of Component (A), but is not limited thereto. The peroxy salt can be an alkali metal salt of a persulfate, a percarbonate, a perborate, and the like, or a salt that is a source of ammonia Preferably, the peroxy salt is selected from ammonium persulfate, sodium persulfate, potassium persulfate, and mixtures thereof. The total amount of peroxy salt, based on the weight of Component (A) may be in the range of about 20 to about 75 weight percent, preferably in the range of about 25 to about 60 parts by weight percent.

Component (A) also optionally includes at least one water-soluble or water-dispersible cosmetic adjuvant. Water-soluble cosmetic adjuvants, when present, are preferably substantially anhydrous, and may be a flow control agent, an auxiliary hair conditioning agent, a wetting agent, a viscosity adjusting agent, a pH adjusting agent, a product finishing agent, and the like, and mixtures thereof. Those skilled in the formulation arts will recognize that ingredients in a formulation can serve more than one function.

The term “flow control agents” as used herein refers to compounds that provide binding and/or anticaking properties to keep Component (A) in a free-flowing, homogeneous state prior to being mixed with Component (B). Flow control agents known in the art include, without limitation, binder agents, such as gums, polymers, starch (e.g., corn (Zea Mays) starch), alkylene glycols (e.g., PEG, PPG, and copolymers thereof), polyalkyleneimines (e.g., polyethyleneimines), and the like, and mixtures thereof. Some binder agents, such as gums, polymers, and starches may also function as viscosity modifying agents when Component (A) is mixed with an aqueous liquid medium. Anticaking agents well known in the art include, without limitation, stearate soaps, such as sodium stearate, aluminum stearate, calcium stearates, and the like; silica (e.g., pyrogenic silica); magnesium carbonate, silicates (e.g., magnesium aluminum silicate), and mixtures thereof.

Auxiliary hair conditioning agents preferably include, without limitation cationic polymers, such as homopolymers of dimethyldiallylamrnmonium salts, preferably chloride salts (DMDAC), (e.g., polyquaternium-6), copolymers of DMDAC (e.g., polyquaternium-4), cationic cellulose derivatives, (e.g. polyquaternium-10), quaternized vinylpyrrolidone/vinylimidazole polymers, cationic guar gum, cationic polymers having at least one acrylamide unit or acrylamide derivative (e.g., polyquaternium-7, and copolymers of trimethylammonioethyl methacrylate chloride and acrylamide), and the like. Polyquaternium-6 in substantially, anhydrous powder form is particularly preferred. Auxiliary hair conditioning agents may also be monomeric quaternary ammonium compounds and salts thereof well known in the cosmetics arts. Auxiliary hair conditioning agents, when present, are preferably in the range of about 1 to about 5 weight percent of the weight of Component (A).

Viscosity modifying agents include, but are not limited to, substantially anhydrous, water-soluble thickeners selected from the group consisting of cellulose ethers, starches, gums, polymers, and the like, and combinations thereof. Exemplary cellulose ethers include methyl cellulose, ethyl cellulose, hydroxyethyl cellulose, methylhydroxyethyl cellulose, methylhydroxypropyl cellulose and carboxymethyl cellulose; exemplary starches include corn starch; starch ethers including carboxymethyl starch, hydroxyethyl starch and methyl starch; exemplary gums include guar gum, xanthan gum, alginates; and exemplary polymers include cationic polymers, such as cationic cellulose derivatives, cationic starch, and anionic polymers in substantially anhydrous form. Preferred water-soluble thickeners are xanthan gum, hydroxyethylcellulose, and mixtures thereof. The amount of thickener can be readily determined by the skilled formulator based on the viscosity desired when Component (A) is admixed with Component (B).

Wetting agents include, but are not limited to, nonionic surfactants, anionic surfactants, cationic surfactants, amphoteric surfactants (including zwitterionic surfactants), and mixtures thereof, so long as they are in substantially anhydrous liquid or solid form. Wetting agents are well known in the art as any of a group of surface active agents which when added to a liquid, cause the liquid to spread more easily over, or penetrate into, a substantially solid surface, and are commercially available from numerous sources found in the trade literature. When present, wetting agents are preferably in the range of about 0.5 to about 10 weight percent, based on the weight of Component (A).

Component (A) preferably provides a pH in the range of at least about pH 8 to about pH 11, when Component (A) is mixed with Component (B) for use. Component (A) preferably includes an effective amount of alkalizing agent, either in the self-emulsifying, fatty acid-derived conditioner or includes a pH adjusting additive, to provide the desired pH when Component (A) is mixed with Component (B). Exemplary pH adjusting agents include, without limitation, alkali metal salts, such as sodium metasilicate, sodium carbonate, ammonium bicarbonate, and the like, and mixtures thereof. Those skilled in the art will recognize that a buffering agent, such as sodium bicarbonate, an acidifying agent, such as organic acid in substantially anhydrous form, and the like, can be employed to achieve a desired pH in the final composition, if necessary.

Exemplary product finishing agents include, without limitation, chelating agents for heavy metals, e.g. salts of ethylenediaminetetraacetic acid, as well as cosmetically acceptable colorants known in the art, such as an ultramarine dyestuff, an acidic dyestuff, a lake, a pigment, and the like.

Those skilled in the formulation arts will recognize that the usefulness and actual amounts of individual ingredients or combination thereof is limited only by the stability of the ingredient in the formulation during storage, and in the presence of oxidizing agent during use when Component (A) is mixed with Component (B). The actual amounts of individual ingredients or combination thereof and water-soluble cosmetic adjuvants can be readily determined by the skilled formulator based on the properties desired in Component (A) and Component (B) separately and when admixed for use.

Component (B) preferably is an aqueous liquid medium containing a concentration of hydrogen peroxide in the range of about 1 to about 12 weight percent, more preferably in the range of about 3 to about 6 weight percent (i.e., such as 10 to 20 volume hydrogen peroxide), on a total liquid Composition (B) weight basis. Commercially available liquid hydrogen peroxide is generally stabilized to a pH in the range of about 3 to about 4, as is well known in the art. A preferred Component (B) may be provided in the form of a hydrogen peroxide-containing emulsion typically referred to in the hair coloring arts as a “creme developer”. Alternatively, a substantially anhydrous powder form of a hydrogen peroxide source may be provided, such as urea peroxide, sodium perborate, sodium carbonate peroxide, and the like, which is dissolved in water to provide a hydrogen peroxide containing liquid medium.

For lightening hair under relatively high alkaline conditions, i.e., pH of at least about 9, and especially at a pH of at least about 10, either one or both of Component (A) or Component (B) preferably may contain a hair protectant, deswelling agent, such as described in U.S. Pat. No. 5,348,737, No. 5,639,449, No. 5,641,477, and No. 5,756,077, all to Syed, et al., the disclosures of which are incorporated herein by reference. A preferred hair protectant, deswelling agent is a polyol including, without limitation, a carbohydrate, such as a starch hydrolysate; a sugar; (e.g., sucrose, glucose, fructose, and the like), an aliphatic polyol, (e.g., glycerol, propylene glycol, and the like), a sugar alcohol, (e.g., sorbitol, maltitol, and the like), and mixtures thereof. Particularly preferred carbohydrate deswelling agents are starch hydrolysates, such as hydrogenated starch hydrolysate sold in various grades under the trade name HYSTAR® (e.g., HYSTAR® HM-75, HYSTAR® 7000, HYSTAR® 6075, and HYSTAR® CG) or as spray-dried polyglycitol powder under the trade name STABILITE™ (e.g., STABILITE™ SD30, and SD60); and maltodextrin having a dextrose equivalence (DE) of less than 20, such as the spray-dried, cold water-soluble glucose polymers reportedly produced by the controlled depolymerization of corn starch sold under the trade name MALTRIN®, having a DE in the range of about 4 to about 19.5 (e.g., MALTRIN® grades M040, M050, M070, M100, M150, and M180).

A presently preferred Component (A) contains a deswelling agent, preferably a maltodextrin or hydrogenated starch hydrolysate. A presently preferred Component (B) is a hydrogen peroxide-containing emulsion including a starch hydrolysate, commercially available under the trade name AFFIRM® MoisturColor® Moisturizing Creme Developer from Avlon Industries, Inc. A preferred conditioning hair lightener emulsion is prepared with each of the foregoing Components (A) and (B) combined at a weight ratio in the range of about 1:1.5 to about 1:2 substantially immediately before use.

Another preferred hair protectant, deswelling agent is a cationic polymer. A particularly preferred cationic polymer is described in U.S. Pat. No. 5,756,077 as the reaction product of a dialkylamine (where the alkyl group has 1 to 3 carbon atoms), and a difunctional epoxy-type reactant and a third reactant selected from the group consisting of ammonia, primary amines, alkylenediamines having two to six carbon atoms in the alkylene group and polyamines, such as the product of a condensation reaction of ethylenediamine, dimethylamine and epichlorohydrin (commercially available under the trade name Betz® Polymer 1195). The cationic polymer is described in more detail in U.S. Pat. No. Re. 28,808 to Panzer et al., (See, for instance, Example 2 of the '808 reissue patent), and U.S. Pat. No. 4,661,259 to Walterick et al., the relevant disclosures of which are incorporated herein by reference.

Component (A), on being mixed with Component (B), preferably in a weight ratio of (A):(B) in a range of about 1:1 to about 1:4, more preferably in a range of about 1:2 to about 1:3, provides a non-runny, conditioning hair lightener emulsion substantially immediately, i.e., within about five minutes, preferably within about 3 minutes, more preferably within about 2 minutes, with minimal manual mixing agitation, such as by stirring or shaking at ambient room temperature. The viscosity of the resultant hair lightener emulsion is not limited as long as the emulsion remains in contact with, and does not drip from, the hair during the hair lightening process. A suitable viscosity is at least about 30,000 mPa·s (Brookfield viscometer, Model No. RVT, Helipath Spindle C, at 10 revolutions per minute), but is not limited thereto. A particularly preferred conditioning hair lightener emulsion embodiment for lightening hair at a relatively high alkaline pH is in the form of a cream or paste having a viscosity in the range of about 65,000 to about 80,000 mPa·s, and a pH of at least about 9, more preferably in the range of about 10 to about 10.5.

The hair lightener system of this invention may also include a post-lightening acidic conditioner, which preferably is an aqueous composition having a pH value of not more than about pH 5, more preferably in the range of about pH 3 to about pH 4.

The post-lightening acidic conditioner may be a hair protectant composition of the type described in U.S. Pat. No. 5,756,077 to Syed, et al., the disclosures of which are incorporated herein by reference.

A preferred post-lightening acid conditioner embodiment includes at least one of a nonionic polymer thickener, a water-dispersible, cationic polyquaternary polymer, and the like, such as described in U.S. Pat. No. 5,756,077, No. 5,348,737, and No. 5,639,449, all to Syed, et al., the relevant disclosures of which are incorporated herein by reference.

A preferred polymeric thickener is polyvinylpyrrolidone (PVP), preferably having a K value of about 90, or a PVP derivative, such as vinylpyrrolidone/dialkylaminoalkyl acrylate or methacrylate copolymers (quaternized or unquaternized), (e.g. such as those sold under the name GAFQUAT®), but is not limited thereto.

The inventive hair lightener system can also include a post-lightener cationic hair conditioner, containing at least one of a polymeric or non-polymeric quaternary ammonium hair conditioning compound or salts thereof.

The hair lightener system of this invention may also include a post-lightening shampoo, preferably having a pH in the range of about 4 to about 6. A particularly preferred post-lightening shampoo contains sufficient free acid to substantially neutralize any residual alkalinity in the lightened hair, such as are conventionally referred to in the art as a “normalizing” type shampoo.

Component (A) can be manufactured by methods known in the art for blending substantially dry ingredients under manufacturing conditions that avoid dampness, such as by employing a powder mixer, such as a ribbon blender, a mechanical fluidized bed mixer, a V-blender, a conical mixer, and the like. A preferred method is to prepare a mixture of the peroxy salt portion with the cosmetic adjuvant portion, when present, and then add the self-emulsifying, fatty acid-derived conditioner portion to provide Component (A). Preferably, the self-emulsifying, fatty acid-derived conditioner portion is in a liquid form, in which case, one or more liquid self-emulsifying fatty acid-derived conditioner ingredients can be premixed to provide a liquid blend. Where one or more self-emulsifying fatty acid-derived conditioner is in a non-liquid form, such as a flake, powder, or pellet, and one or more of self-emulsifying fatty acid-derived conditioner is a liquid, the non-liquid conditioner ingredient can be included in the liquid (i.e., dissolved or dispersed in the liquid, heating if necessary), or can be included in the premixed peroxy salt containing portion. The method of manufacturing is unlimited as long as a substantially free flowing Component (A) is obtained. Component (A) may be ground using powder grinding units and powder sifters, known in the art, to obtain a desired average particle size. Preferably Component (A) is sufficiently granular to be free-flowing, with minimal dusting, and dissolve substantially immediately in Component (B).

Component (A) and Component (B) are maintained in separate packages and, substantially immediately before use, Component (B) is admixed with Component (A) to provide a conditioning hair lightener emulsion. One method aspect of lightening the natural color of hair with the conditioning hair lightener system comprises the steps of:

(i) contacting substantially dry hair in need of lightening,with the conditioning hair lightener emulsion provided by a mixture of Components (A) and (B), and distributing the hair lightener emulsion therethrough;

(ii) maintaining the hair lightener emulsion in contact with the hair for a period sufficient to visibly lighten the natural color of the hair by at least one level, or to a desired shade level, to provide lightened hair, and

(iii) removing the hair lightener emulsion from the lightened hair.

In a preferred method aspect, step (iii) is performed by rinsing the hair with water, having a tepid temperature.

In another preferred method aspect of the hair lightening system, the lightened hair is contacted with a post-lightening, acidic aqueous medium having a pH of not more than about 5, preferably having a pH in the range of about 3 to about 4, either substantially immediately after step (ii), concurrently with, or after step (iii). The contact with the acidic medium is maintained for a period of at least about one minute or a period sufficient to substantially minimize or stop any further alkaline or oxidative action of the emulsion on the lightened hair. Preferably a post-lightening acidic conditioner of the type discussed above is employed. Alternatively, the post-lightening, acidic aqueous medium may be an aqueous solution of a cosmetically acceptable organic acid, such as acetic acid, citric acid, tartaric acid, malonic acid, and the like.

In a preferred method aspect, the further step (iv) of washing the hair is practiced with a post-lightening shampoo preferably having a pH in the range of about 4 to about 6. Another preferred method aspect includes contacting the lightened hair with a post-lightener cationic hair conditioner between step (iii) and step (iv) and removing the cationic hair conditioner with water.

The ameliorative effect of a conditioning hair lightener of this invention on the tensile properties of oxidatively lightened hair can be evaluated instrumentally, as well as subjectively, by one or more of the following methods.

One preferred method of evaluating the elastic tensile strength of wet hair is referred to as the “Intermittent Stress Relaxation (ISR) Method” using a Dynamic Mechanical Analyzer (e.g., TA Instruments, Model Q800) equipped with a hair-fiber anchoring assembly that can be submerged in water. In the ISR Method, an individual hair fiber is clamped at two opposing portions in the fiber anchoring assembly, which is then submerged in water. The length of the submerged hair fiber is then stretched in water to a constant strain, such as 0.5% of its length for a period of time, such as about 30 seconds, and allowed to relax by removing the strain for a period of time, such as about 30 seconds, and this cycle of intermittent stress strain and relaxation is repeated for a total of about 10 cycles. The amount of stress (in grams/denier fiber) is measured and recorded before the hair fiber is given a hair lightening treatment, and after the hair lightening procedure. The change in the ratio (index) of the stress of the hair before lightening relative to the stress of the hair after hair lightening is indicative of loss or amelioration of loss in tensile strength from the chemical treatment. An index value of 1=no change in hair strength, an index value of less than 1 indicates a loss (weakening) in tensile strength, and an index value of greater than 1 indicates an increase (strengthening) in tensile strength. Measuring the elasticity of wet hair when stretched in the range of about 0.5 to about 1 percent of its length, is judged as simulating the range of strain applied during a conventional hair grooming process, such as combing and brushing of the wet hair.

Another preferred method for evaluating the wet elasticity of hair is referred to as the “Creep Recovery Method” which instrumentally measures the recovery of the length of a hair fiber after being stretched to a constant force, using a Dynamic Mechanical Analyzer (e.g., Perkin Elmer, Model 7e, with Pyris software) equipped with a hair-fiber anchoring assembly that can be submerged in water. In the Creep Recovery Method, an individual hair fiber is clamped at two opposing portions in the fiber anchoring assembly which is then submerged in a beaker (about 100 ml capacity) containing about 90 ml water. The submerged fiber is then stretched to a constant force, such as 100 milliNewtons (mN) for about 0.1 minute, the force is released and the fiber is allowed to recover (relax) to a force of about 5 mN for about 0.9 minutes, and this cycle of stretch and recovery is repeated for a total of about 15 cycles, while recording the creep recovery curve. A baseline creep recovery curve is measured and recorded in this manner from a hair fiber before being lightened, thereafter the hair fiber is removed from the instrument, allowed to dry (for at least about one hour at ambient room temperature), the dried hair fiber is given a hair lightening treatment and the creep recovery curve of the lightened hair is measured and recorded as previously described. An index value for the creep recovery of the wet hair after lightening relative to the creep recovery of the hair before lightening is calculated from the area of the curves. An index value of 1=no change in hair strength, an index value of less than 1 indicates a toughening or strengthening of the hair, and an index value of greater than 1 indicates a weakening of the hair.

Another preferred method of evaluating amelioration is to determine changes in the alkali solubility of the lightened hair by the well known “Alkali Solubility Test” method. A description of the Alkali Solubility Test is found in Edman, et al., “Properties of Peroxide-Bleached Hair,” Journal of the Society of Cosmetic Chemistry, 12, 133-145 (1961). An increase in the alkali solubility of keratin (such as hair and wool) has been recognized in the cosmetic and textile arts as being indicative of undesirable degradation of the protein structure from chemical treatments. The alkali solubility of lightened hair is evaluated by first subjecting an intact hair tress to an alkaline hair lightener procedure for a given period of time, removing the hair lightener with water, drying the lightened hair, equilibrating the lightened hair to a constant weight, and recording the constant dry weight (initial weight) of the equilibrated, lightened hair. The equilibrated, lightened hair is then subjected to an alkali treatment by immersing the hair in a bath of 0.1N sodium hydroxide for a period of time (about one hour), heated to a temperature in the range of about 60 to about 63° C. The alkali-treated lightened, hair is then removed from the bath, rinsed with water and dilute acid, dried, equilibrated to a constant weight, and the equilibrated weight (final weight) is recorded. The alkali solubility is calculated from the change in the weight of the alkali-treated, lightened hair tress relative to the initial weight of the lightened hair tress (i.e., before alkaline treatment) and expressed in terms of a percentage of the change (i.e., loss). Alkali solubility=(initial weight of lightened hair−final weight of alkali-treated lightened hair)×100/initial weight of lightened hair.

Still another preferred method is to determine the change in porosity of the hair after a lightening procedure by determining the change in the weight amount of absorbed water that is retained by the lightened hair. This “weight” method is well known in the art as “Liquid Retention” or “Porosity Test,” and the technique has been found to correlate chemical hair damage with loss of tensile strength. A description and discussion of the Liquid Retention procedure is found in Ali N. Syed, et al., “Correlating Porosity and Tensile Strength of Chemically Modified Hair,” Cosmetics & Toiletries, 117 (11), 57-64, (2002). Hair that has been subjected to a hair lightening procedure is initially equilibrated at an ambient room temperature in the range of about 26 to about 27° C in a chamber having a relative humidity of about 65% for a selected time period and the initial weight (W) of the equilibrated lightened hair is determined with a microbalance. The equilibrated lightened hair is then immersed in deionized water for a selected time, after which the water-soaked hair is removed from the water, placed in centrifuge tubes containing a mesh at the bottom to keep the water-swollen hair from contact with draining water. The water-swollen, lightened hair is centrifuged to drain off all externally imbibed water, and the weight (Wa) of the water-swollen lightened hair immediately after centrifuging is determined. The percent liquid retention (porosity) is then calculated using the following formula:

([Wa−(W−0.162W)]/Wa)×100;

where Wa=weight of water-swollen lightened hair immediately after centrifuging; W=initial weight of lightened hair at 65% R.H.; and (0.162 W) represents the average % moisture reportedly absorbed from 0% to 65% R.H. for normal hair. The amount of water retained by the lightened hair relates to the swelling property (i.e., porosity) of the hair, so the higher the amount of liquid retained, the greater the porosity or chemical alteration of the hair.

The ease of combing (wet or dry) of tresses before and after lightening can be instrumentally evaluated using an automated Dia-Stron mini tensile tester equipped with Uvwin software. In this procedure, each tress is combed before and after the lightening treatment and the amount of energy or work (joules) required to comb each tress is measured and recorded. The tresses are equilibrated at an ambient room temperature in the range of about 26 to about 27° C. in a chamber having a relative humidity of about 65% for a period of about 24 hours before each combing. The tresses are glued to a tab at the hair root portion, and the tabbed portion is secured to a tress anchor jaw unit of the tester and combed with a hard plastic comb downward from the root to end portion at a rate of about 120 millimeters (mm) per minute. Multiple (preferably 10) consecutive combing readings are taken for each tress. The highest force or peak load (grams-force) is used to measure the relative ease to detangle the hair during combing. A ratio of the reading before the lightening procedure relative to the reading after the lightening procedure is calculated to provide a combing index and used to assess the combing properties of the hair. A ratio value of less than 1 indicates easier combing.

The conditioning hair lightener system is preferably provided in kits, with Component (A) in packaged form together with instructional indicia for admixing Component (A) with Component (B) for use. A preferred kit embodiment also includes Component (B), and, optionally, at least one of a post-lightening acidic conditioner, a post-lightening cationic conditioner, and a post-lightening shampoo, each in separately packaged form, and one or more of the following implements for performing the hair lightening process, i.e., disposable gloves, measuring scoop for Component (A), mixing tools, e.g., a mixing spatula, a mixing vessel, and the like. Preferably the hair lightening implements are substantially chemically unreactive with the oxidative ingredients in the hair lightener emulsion. The kit components preferably are contained in an outer package. The outer package can be a box or carbon or shrink wrap, and preferably has instructional indicia printed thereon or visible therethrough.

The components of the hair lightener system can be provided in amounts suitable for a single application or in sufficient amounts for multiple applications, and can be readily determined by those skilled in the art.

Useful instructional indicia can be printed media, aural media, visual aids, electronic media or a combination thereof, which instruct the user on how to admix the Component (A) with Component (B) and describe the use of the hair lightener emulsion product to lighten the hair. Printed media includes, but is not limited to, labels attached to or imprinted on the components of the kit, package inserts, pamphlets, books, flyers, and the like. Aural media includes, but is not limited to, tape recordings, audio compact disks, records, and the like. Visual aids include, but are not limited, to photographs, slides, movies, videos, DVDs, and the like. Electronic media includes all forms of electronic data storage media, such as, but not limited to, diskettes, interactive CD-ROMs, interactive DVDs, and the like.

The following examples further illustrate the preparation and use of preferred embodiments hair lightening, but are not intended to be limited thereto.

### EXAMPLE 1

Substantially anhydrous compositions for use in Component (A) are illustrated by embodiment range (1-A) and the specific embodiment Examples (1-B1) and (1-B2) shown in Table 1 prepared from a peroxy salt-containing powder portion (P) and a self-emulsifying, fatty acid-derived conditioner (SFC) portion illustrated in Table 2. Also shown in Table 1 are specific preferred powder portion (P) Examples (1-B3) and (1-B4), which can be mixed with varying amounts of an (SFC) portion for obtaining preferred embodiments of Component (A), and as indicated in the following examples. Additionally, portion (P) Examples (1-B3) and (1-B4) containing no (SFC) portion can provide a comparative hair lightener emulsion, where so indicated in the following examples.

TABLE 1

| Ingredient | Parts by Weight (As Supplied) | | | | |
|---|---|---|---|---|---|
| (INCI/Common Name) | (1-A) | | | | |
| Portion (P) | Range | (1-B1) | (1-B2) | (1-B3) | (1-B4) |
| 1. Potassium persulfate | 15-40 | 29.4 | 29.4 | 29.7 | 29.7 |
| 2. Sodium persulfate | 5-25 | 14.7 | 14.7 | 14.9 | 14.9 |
| 3. Ammonium | 3-15 | 8.8 | 8.8 | 8.9 | 8.9 |
| persulfate | | | | | |
| 4. Tetrasodium EDTA | 0-0.5 | 0.3 | 0.3 | 0.3 | 0.3 |
| 5. Xanthan gum | 0.1-2 | 0.8 | 0.8 | 0.7 | 0.8 |
| 6. Hydroxyethyl | 0.4-4 | 2.5 | 2.5 | 2.2 | 2.5 |
| cellulose | | | | | |
| 7. Polyquaternium-6 | 0-4 | 3 | — | 3 | 3 |
| (Note 1) | | | | | |
| 8. Silica | 3-15 | 8.8 | 8.8 | 8.9 | 8.9 |
| 9. Cocamide MEA | 0-3 | 1.5 | 2 | 1.5 | 1.5 |
| 10. Sodium stearate | 2-12 | 7.1 | 7.5 | 7.4 | 7.2 |
| 11. Aluminum stearate | 3-15 | 8.8 | 9 | 8.9 | 8.9 |
| 12. Sodium metasilicate | 4-17 | 12.9 | 14.8 | 13.1 | 13 |
| 13. Ultramarine Blue | 0-0.75 | 0.3 | 0.3 | 0.5 | 0.3 |
| (SFC) Portion | 0.1-10 | 1.1 | 1.1 | 0-q.s. | 0-q.s. |

q.s. = quantity sufficient
Note to Table 1.
1. Powder, such as sold under the tradename RHEOCARE ® CC6P

Preferred liquid (SFC) portion embodiments are illustrated in Table 2 by embodiment range Example (1-C), specific embodiment Examples (1-D), (1-E), and (1-F), and embodiment range Examples (1-G), (1-H), and (1-I).

TABLE 2

| | Parts by Weight (as Supplied) | | | | | | |
|---|---|---|---|---|---|---|---|
| Ingredient | (1-C) | | | | (1-G) | (1-H) | (1-I) |
| (INCI/Common Name) | Range | (1-D) | (1-E) | (1-F) | Range | Range | Range |
| Glyceryl cocoate/citrate | 5-25 | 16 | 15 | 18 | 95-99.5 | — | — |
| lactate (Note 1) | | | | | | | |
| PEG-40 sorbitan | 25-40 | 34 | 25 | 37 | — | 95-99.5 | — |
| peroleate (Note 2) | | | | | | | |
| Caprylic/capric | 30-65 | 50 | 40 | 55 | — | — | 95-99.5 |
| triglyceride (Note 3) | | | | | | | |
| Glyceryl stearate SE (Note 4) | 0-5 | — | — | — | 0.5-5 | 0.5-5 | 0.5-5 |

Notes to Table 2.
1. INCI name for a product sold under the tradename INWITOR ® 380, reported to be glycerine esterified with a blend of coconut, citric and lactic acids.
2. INCI name for a mixture of oleic acid esters of sorbitol condensed with an average of 40 moles of ethylene oxide.
3. INCI name for the mixed triester of glycerin and caprylic and capric acid.
4. INCI name for self-emulsifying grade of glyceryl stearate that contains some sodium and/or potassium stearate.

Component (A) can be prepared by the general method of separately preparing a homogeneous blend of a powder portion (P) ingredient nos. 1-13, (as shown in Table 1), and then adding an (SFC) portion (as shown in Table 2), and admixing until the formulation is homogeneous. The (SFC) portion is preferably prepared as a separate blend and then admixed with portion (P).

The (SFC) portion, such as embodiments Examples (1-D), (1-E) and (1-F) can be prepared by mixing the ingredients together at ambient room temperature. The (SFC) portion embodiments Examples (1-G), (1-H) and (1-I) can be prepared by heating the ingredients together to a temperature in the range of about 65 to about 70° C. to dissolve the glyceryl stearate (SE) and then cooling the mixture to ambient room temperature before adding it to the portion (P).

### EXAMPLE 2

Component (A) was prepared from a mixture of about 98.9 parts by weight of portion (P) Example (1-B3) shown in Table 1 and 1.1 parts by weight of (SFC) portion Example (1-D) shown in Table 2. The (SFC) portion was added to portion (P) and admixed until homogeneous and free flowing (no clumps observed). Component (A) was then ground using a powder grinder unit, to a particle size in the range of about 0.3 to about 0.4 millimeters (mm), determined by sifting Component (A) through a sieve having a 30 mesh size (100% pass through), and a 50 mesh size (not more than about 3 to about 6% remaining in sieve).

Component (A) at a concentration of about 1% in deionized water provided a pH in the range of about 10 to about 11. One part by weight of Component (A) admixed with two parts by weight of an aqueous medium containing about 6% hydrogen peroxide, Component (B), having a pH in the range of about 3.5 to about 4, provided a viscous, conditioning hair lightener emulsion having a pH in the range of about 10 to about 10.2, which formed substantially immediately (within about one minute).

Component (A) contained a total SFC content of about 1.1% by weight, based on the weight of Component (A). The conditioning hair lightener emulsion contained about 0.4% SFC based on the weight of the emulsion (i.e., mixture of 1:2 parts by weight Component (A):Component (B)).

### EXAMPLE 3

Post-lightening acidic conditioner embodiments are illustrated in Table 3 by embodiment range Example 3(A) and specific embodiment Examples 3(B), 3(C) and 3(D).

TABLE 3

| | | Parts by Weight (As Supplied) | | | |
|---|---|---|---|---|---|
| | | 3 (A) | | | |
| Ingredient (INCI/Common Name) | | Range | 3 (B) | 3 (C) | 3 (D) |
| 1. | PVP (K90P) | 0.1-2 | 0.2 | 1.2 | 0.1 |
| 2. | Botanical conditioners | 0-3 | 0.5* | — | — |
| 3. | Betz ® Polymer 1195 | 0-2 | 0.5 | — | 1.5 |
| 4. | Preservative | q.s. | q.s. | q.s. | q.s. |
| 5. | pH adjusting agent to pH 3-4 | q.s. | q.s. | q.s. | q.s. |
| 6. | Deionized water to 100 parts | q.s. | q.s. | q.s. | q.s. |
| | by weight (pbw) | | | | |

q.s. = quantity sufficient
*Preferably Aloe Vera, (44 pbw); Actiphyte of Japanese Green Tea (55 pbw)

### EXAMPLE 4

This example illustrates a method of lightening the color of naturally dark brown Caucasian hair using a conditioning hair lightener system and emulsion of this invention with and without employing a post-lightening acidic conditioner step, (Study 4-a and 4-b respectively), under simulated practical use conditions. The effect on the wet strength of the hair was evaluated by the Creep Recovery method as follows.

Study 4-a. A conditioning hair lightening emulsion (I) was prepared by admixing one part by weight of Component (A) of Example 2 with two parts by weight of Component (B), an aqueous hydrogen peroxide (6%) containing product, (AFFIRM® MoisturColor® 20 Volume, Moisturizing Creme Developer, Avlon Industries, Inc.). The creep recovery of the wet hair, before and after lightening with the hair lightening emulsion (I), was instrumentally evaluated by the Creep Recovery Method previously described using the Dynamic Mechanical Analyzer. The average Creep Recovery Index (CRI) for five fibers per study was calculated and the results compared as described below.

An untreated test fiber of about 10 mm length, Caucasian hair (DeMeo Brothers) was submerged in about 90 ml water, and then subjected to a stretch and recovery cycle of being stretched to a constant force of about 100 mN for about 0.1 minutes, relaxed to about 5 mN for a recovery period of about 0.9 minutes, and repeating this stretch and recovery cycle for a total of about 15 cycles, to establish a baseline creep recovery curve. The fiber was then dried in air for at least about one hour at ambient room temperature and humidity.

The dry fiber was then given a hair lightener treatment by: (i) applying a sufficient amount of conditioning hair lightening emulsion (I) to coat the fiber, allowing emulsion (I) to remain in contact with the fiber for a hair lightening period of about 60 minutes; (ii) applying a sufficient amount of post-lightening acidic conditioner (II) of Example 3(B), having a pH in the range of about 3.5 to about 4, directly to the coated, lightened fiber (without an intervening water rinse) and left in contact therewith for about 5 minutes; (iii) rinsing the acidic-conditioned, lightened fiber with tepid tap water for about 3 minutes; (iv) contacting the water-rinsed, lightened fiber with a commercial cationic hair conditioner (AFFIRM® 5 in 1 Reconstructor, Avlon Industries, Inc.) for a period of about 5 minutes; (v) rinsing the cationic hair conditioner from the lightened fiber with water for about three minutes; (vi) washing the rinsed fiber with a non-conditioning, normalizing-type shampoo having a pH in the range of about 4.5 to about 5.5; and (vii) rinsing the shampooed hair with water for about one minute.

The resultant lightened fiber was dried in air for at least about one hour at ambient room temperature and humidity, the procedure for obtaining the Creep Recovery Index (CRI) was repeated, as described above, and the average CRI value was calculated.

Study 4-b. The procedure of study 4-a was repeated, except that the step (ii) of applying the post-lightening acidic conditioner (II) was omitted.

The hair lightened by the procedure of study 4-a had a calculated average CRI value of about 1.25 and the hair lightened by the procedure of study 4-b had a calculated average CRI value of about 1.97, based on five fibers per study. Relative to untreated hair (Index=1), the creep recovery of the lightened hair fibers from study 4-a showed that contacting the lightened hair with the post-lightening acidic conditioner ameliorated the loss in elasticity to a significantly greater extent than lightening the hair without this step.

### EXAMPLE 5

The beneficial effect of a conditioning hair lightener system of this invention on the subjective, tactile and visual, properties of the lightened hair was demonstrated as follows. A conditioning hair lightener emulsion (I) was prepared from 7 grams (g) of Component (A) of Example 2 and 14 grams of aqueous Component (B) containing about 6% hydrogen peroxide (AFFIRM® MoisturColor® 20 Volume, Moisturizing Creme Developer, Avlon Industries, Inc.). Emulsion (I) was applied to a Caucasian hair tress (about 5 g in weight, about 20 cm in length, medium brown in color) (DeMeo Brothers), and left in contact with the hair tress for a period of about 60 minutes, emulsion (I) was removed from the hair by rinsing with tepid tap water, and the lightened tress (Tress I) was air dried at ambient room temperature and humidity conditions for at least about 12 hours. For comparison, a separate similar tress (Tress II) was lightened by repeating the lightening procedure, except that the hair lightener emulsion was prepared with Component (A) of Example (1-B3) containing no (SFC) portion to provide a comparative hair lightener emulsion (II).

The subjective properties of the hair (Tress I) lightened with the conditioning hair lightener emulsion (I) of this invention were evaluated by a panel of seven persons and compared to those of hair (Tress II) lightened with non-conditioning hair lightener emulsion (II). Each panelist evaluated the subjective attributes of ease of combing, degree of conditioning, visual sheen, and lack of static flyaway, for each of the tresses based on a numerical rating scale of 1 to 5, and the average ratings for each of the attributes are shown in Table 4.

TABLE 4

Average Rating
5 = Most Preferred;
1 = Least Preferred)

| Subjective Attribute | (Tress I) | (Tress II) |
|---|---|---|
| Ease of Combing (Dry) | 5 | 3.6 |
| Degree of Conditioning | 4.9 | 3.9 |
| Sheen | 4.9 | 4.7 |
| Lack of Static Flyaway | 5 | 3.6 |

Each of the seven panelists also evaluated the subjective tactile properties (feel) of Tresses (I) and (II) based on the following numerical rating scale of: 1=Brittle, strawlike; 2=Very rough; 3=Rough; 4=Soft; 5=Very soft. Five panelists rated the hair lightened (Tress I) by the conditioning emulsion (I) of this invention, as soft (Average Rating=4) and two panelists rated (Tress I) as very soft (Average Rating=5). In contrast, five panelists rated the hair lightened (Tress II) by the comparative emulsion (II) as rough (Average Rating=3) and two panelists rated (Tress II) as very rough (Average Rating=2).

The results show that the beneficial effects of the conditioning hair lightener of this invention (i.e., containing SFC) on the subjective properties of lightened hair were judged discernibly superior to those of hair lightened with the comparative, hair lightener containing no SFC.

### EXAMPLE 6

This example illustrates the beneficial effect of a conditioning hair lightener of the invention, based on the Alkali Solubility Method, previously described. Caucasian hair tresses (about 5 g in weight, about 20 cm in length, medium brown in color) (DeMeo Brothers) were separately lightened for either about 30 minutes (A), about 45 minutes (B), about 60 minutes (C), or 90 minutes (D), as described in study 6-a and 6-b below.

Study 6-a. A conditioning hair lightener emulsion (I) of this invention was prepared by admixing about 7 grams of Component (A) of Example 2 (containing a total of about 1.1% SFC) with about 14 grams of aqueous Component (B) containing about 6% hydrogen peroxide (AFFIRM® MoisturColor®, 20 Volume, Moisturizing Creme Developer, Avlon Industries, Inc.). Emulsion (I) was applied to a tress and left in contact therewith for the described selected lightening time period, emulsion (I) was removed from the tress by rinsing with tepid tap water, and the lightened tress was dried in air for at least about 12 hours at ambient room temperature and humidity.

The air-dried lightened hair tress was cut into small pieces. About 1 gram weight sample of the hair cuttings was placed in an aluminum weighing dish and heated in an oven set at a temperature of about 105° C. for about 2 hours, cooled to ambient room temperature in the range of about 26 to about 27° C. in a chamber having a relative humidity of about 65%, and weighed to obtain a constant initial dry weight for the lightened hair. The weighed hair sample was then transferred to an Erlenmeyer flask (250 ml capacity) containing about 100 ml of 0.1N NaOH (preheated to a temperature in the range of about 60 to about 63° C.). The flask was sealed with a cling wrap film to exclude air. The flask contents were heated for about 60 minutes at a temperature in the range of about 60 to about 63° C. to provide alkali-treated, lightened hair. The alkali-treated hair sample was then filtered (Whatman paper filter), and sequentially washed six times with deionized water (about 50 ml/washing), washed once with about 50 ml aqueous acetic acid (1%), washed six times again with deionized water (about 50 ml/washing), and then dried in air for at least about 12 hours at ambient room temperature and humidity. The air dried alkali-treated, lightened hair sample was then heated in an oven set at a temperature of about 105° C. for about 2 hours, cooled to ambient room temperature in the range of about 26 to about 27° C. in a chamber having a relative humidity of about 65%, and then weighed to obtain a final constant weight.

The alkali solubility was calculated from the loss in the weight of the lightened hair after the alkaline procedure, expressed as a percentage of the initial weight of the hair (i.e., before alkaline treatment). Thus, alkali solubility=(initial weight of lightened hair−final weight of alkali-treated lightened hair)×100/initial weight of lightened hair. The average percent alkali solubility, based on three separate samples per lightening time period, is shown in Table 5.

Study 6-b. The procedure of study 6-a was repeated, except that the hair lightener emulsion was prepared by admixing about 7 grams of portion (P) of Example (1-B3) containing no (SFC) portion with about 14 grams of the Component (B) described above to use as Component (A) to provide a comparative hair lightener emulsion (II).

The average % alkali solubility of the tresses from study 6-a (Tress (I-A)-(I-D)) and the tresses from Study 6-b (Tress (II-A)-(II-D)) are shown in Table 5, compared to the average % alkali solubility for unlightened hair (control).

TABLE 5

| | Emulsion | Lightening | Alkali Solubility |
|---|---|---|---|
| Lightening Treatment | Contains SFC | Time (Min.) | Average % |
| Control, None | — | None | 7.7 |
| Study 6-a, Tress (I-A) | Yes | 30 | 9.6 |
| Study 6-b, Tress (II-A) | No | 30 | 10.3 |
| Study 6-a, Tress (I-B) | Yes | 45 | 16 |
| Study 6-b, Tress (II-B) | No | 45 | 21.6 |
| Study 6-a, Tress (I-C) | Yes | 60 | 16.5 |
| Study 6-b, Tress (II-C) | No | 60 | 24 |
| Study 6-a, Tress (I-D) | Yes | 90 | 23 |
| Study 6-b, Tress (II-D) | No | 90 | 26.7 |

The level of hair lightening achieved on Tress (I) and Tress (II) was judged substantially equivalent. The results show that the conditioning hair lightener emulsion (I), which contained about 0.4% SFC, beneficially diminished the alkali solubility of the lightened hair at all times compared to that of hair lightened with hair lightener Emulsion (II containing no SFC.

### EXAMPLE 7

This example illustrates the beneficial effect of a conditioning hair lightener of this invention by the Intermittent Stress Relaxation (ISR) Method previously described.

Study 7-a. One part by weight Component (A) of Example 2 was mixed with two parts by weight of aqueous Component (B), containing about 6% hydrogen peroxide (AFFIRM® MoisturColor® 20 Volume, Moisturizing Creme Developer, Avlon Industries, Inc.) to provide a conditioning hair lightener emulsion (I) of this invention containing about 0.4% by weight SFC.

A tress was prepared of Caucasian hair (about 1 g in weight), naturally brown in color, (about 20 cm in length) (DeMeo Brothers) in which the hair fibers were mixed to provide a random blend of root-to-tip and tip-to-root orientation. About a 10 cm portion of the hair tress (referred to as the upper portion) was secured within a plastic tube to prevent contacting the upper portion of the hair tress with the hair lightener emulsion. The remaining exposed portion of the hair tress (referred to as the lower portion) was then contacted with a sufficient amount of conditioning hair lightener emulsion (I) to substantially uniformly coat the tress and lightened for about 60 minutes.

The conditioning hair lightener emulsion (I) was removed from the hair by rinsing the hair with tepid tap water for about 3 minutes. The rinsed hair was then coated with a commercial cationic hair conditioner (AFFIRM® 5 in 1 Reconstructor, Avlon Industries, Inc.) for about 5 minutes, followed by rinsing with tepid tap water for about 3 minutes, shampooing with a non-conditioning, normalizing-type shampoo having a pH in the range of about 4.5 to about 5.5 and then rinsed for about one minute with tepid tap water.

The ISR was evaluated for eight fibers randomly sampled from the hair tress by measuring the ISR of the untreated portion and the lightened portion of the same fiber and then calculating the ISR Index in this manner for each of the fibers.

Study 7-b. The procedure of study 7-a was repeated except that the hair lightener emulsion was prepared with one part by weight of portion (P) Example (1-B3) containing no SFC as Component (A) to provide a comparative hair lightener emulsion (II).

Study 7-c. The procedure of study 7-a was repeated except that Component (A) was Example (1-B1) prepared with (SFC) portion (1-D) to provide hair lightener emulsion (III).

Relative to untreated hair (ISR Index=1) the average ISR Index for fibers from Study 7-a and 7-c was in the range of about 0.84 to about 0.89 and for fibers from Study 7-b was about 0.79. The results show that the conditioning hair lightener emulsions (I) and (III) containing about 0.4% SFC ameliorated the loss in elasticity of the fibers to not more than 20% (e.g., a range of about 11% to about 16%), whereas the hair lightener emulsion (II) containing no SFC represents a loss of more than 20% (e.g., about 21%) elasticity.

### EXAMPLE 8

Preferred conditioning compositions containing hair protectant deswelling agents for use in Component (A) of the hair lightener system are illustrated in Table 6 by broad range embodiments Example (8-A) and preferred range embodiment Example (8-B) having a peroxy salt-containing portion (P) and a self-emulsifying, fatty acid-derived conditioner (SFC) portion illustrated in Table 7.

TABLE 6

| Ingredient | | Parts by Weight (As Supplied) | |
|---|---|---|---|
| (INCI/Common Name) | | (8A) | (8B) Preferred |
| Portion (P) | | Broad Range | Range |
| 1. | Potassium persulfate | 10-50 | 15-40 |
| 2. | Sodium persulfate | 5-30 | 10-25 |
| 3. | Ammonium persulfate | 2-20 | 3-15 |
| 4. | Tetrasodium EDTA | 0-1.5 | 0.05-1 |
| 5. | Viscosity Modifying Agent (Note 1) | 1-10 | 0.05-8 |
| 6. | Polyquaternium-6 (Note 1, Table 1) | 0.5-5 | 1-4 |
| 7. | Deswelling Agent (Note 2) | 2-25 | 5-20 |
| 8. | Flow Control Agent (Note 3) | 0.5-20 | 1-15 |
| 9. | Sodium metasilicate | 3-30 | 4-25 |
| 10. | Wetting agent (Note 4) | 0-10 | 0.5-5 |
| 11. | Ultramarine Blue | 0-0.75 | 0.1-0.5 |
| 12. | (SFC) Portion | 0.1-5 | 0.5-1.5 |
| 13. | Sunflower oil | 0-10 | 1-5 |
| 14. | Octyl pelargonate | 0-10 | 1-5 |

Notes to Table 6.
1. Preferably xanthan gum, hydroxyethyl cellulose, and mixtures thereof.
2. Preferably maltodextrin, hydrogenated starch hydrolysate, or mixture thereof.
3. Preferably silica, starch, or mixture thereof.
4. Preferably a powder form cationic surfactant, nonionic surfactant, anionic surfactant or mixture thereof.

Liquid (SFC) portion embodiments are illustrated in Table 7 by broad range embodiment Example (8-C), preferred range embodiment Example (8-D) and specific embodiment Example (8-E).

TABLE 7

| | Parts by Weight (As Supplied) | | |
|---|---|---|---|
| Ingredient | (8-C) | (8-D) | |
| (INCI/Common Name) | Broad Range | Preferred Range | (8-E) |
| Glyceryl cocoate/citrate/lactate | 2-30 | 5-20 | 17 |
| (Note 1, Table 2) | | | |
| PEG-40 sorbitan peroleate | 30-50 | 35-45 | 33 |
| (Note 2, Table 2) | | | |
| Caprylic/capric triglyceride | 40-60 | 45-55 | 50 |
| (Note 3, Table 2) | | | |

Varying amounts of the portion (P) and (SFC) portion can be mixed to provide the desired amount of total SFC in Component (A). A preferred total amount of SFC in Component (A) is in the range of about 0.5 to about 2 weight percent, more preferably in the range of about 1 to about 1.5 weight percent, based on the weight of Component (A).

Component (A) can be prepared by the general method of separately preparing a homogeneous blend of the non-liquid portion (P) ingredient nos. 1-3, and of non-liquid ingredient nos. 4-11, admixing the two homogeneous blends with one another with stirring agitation until homogeneous, and then slowly adding the liquid (SFC) portion (ingredient no. 12) and liquid ingredient nos. 13 and 14, admixing until the Component (A) formulation is homogeneous. The (SFC) portion is preferably prepared as a separate blend of the three ingredients, and then admixed with Portion (P). Ingredient nos. 13 and 14 are auxiliary conditioning agents and preferably are also preblended with one another and added to the formulation either after adding the (SFC) portion, or concurrently with the (SFC) portion.

### EXAMPLE 9

Conditioning compositions, Examples 9-(A-H), for use as Component (A) were prepared containing as hair protectant deswelling agent a maltodextrin of varying Dextrose Equivalence (DE) values as shown in Table 8, suitable for use as conditioning hair lighteners when mixed with hydrogen peroxide-containing compositions.

TABLE 8

| Ingredient | Parts by Weight (As Supplied) | | | | | | | |
|---|---|---|---|---|---|---|---|---|
| (INCI/Common Name) | (9-A) | (9-B) | (9-C) | (9-D) | (9-E) | (9-F) | (9-G) | (9-H) |
| 1. Potassium persulfate | 29.4 | 29.4 | 29.4 | 29.4 | 29.4 | 29.4 | 29.4 | 29.4 |
| 2. Sodium persulfate | 14.7 | 14.7 | 14.7 | 14.7 | 14.7 | 14.7 | 14.7 | 14.7 |
| 3. Ammonium persulfate | 8.8 | 8.8 | 8.8 | 8.8 | 8.8 | 8.8 | 8.8 | 8.8 |
| 4. Tetrasodium EDTA | 0.3 | 0.3 | 0.3 | 0.3 | 0.3 | 0.3 | 0.3 | 0.3 |
| 5. Xanthan gum | 0.8 | 0.8 | 0.8 | 0.8 | 0.8 | 0.8 | 0.8 | 0.8 |
| 6. Hydroxyethyl cellulose | 2.5 | 2.5 | 2.5 | 2.5 | 2.5 | 2.5 | 2.5 | 2.5 |
| 7. Polyquaternium-6 (Note 1, | 3 | 3 | 3 | 3 | 3 | 3 | 3 | 3 |
| Table 1) | | | | | | | | |
| 8. Maltrin ® M040 (Note 1) | 19.4 | — | — | — | — | — | — | — |
| 9. Maltrin ® M050 (Note 1) | — | 19.4 | — | — | — | — | — | — |
| 10. Maltrin ® M100 (Note 2) | — | — | 5 | 16.4 | 19.4 | — | — | — |
| 11. Maltrin ® M150 (Note 3) | — | — | — | — | — | 19.4 | — | — |
| 12. Maltrin ® M180 (Note 4) | — | — | — | — | — | — | 19.4 | 10 |
| 13. Silica | 6 | 6 | 6 | 6 | 6 | 6 | 6 | 8 |
| 14. Corn (Zea Mays) starch | — | — | 11.4 | — | — | — | — | — |
| 15. Sodium metasilicate | 8.8 | 8.8 | 8.8 | 8.8 | 8.8 | 8.8 | 8.8 | 16.2 |
| 16. Ultramarine Blue | 0.3 | 0.3 | 0.3 | 0.3 | 0.3 | 0.3 | 0.3 | 0.3 |
| 17. (SFC) portion of Ex. 8E, | 1 | 1 | 1 | 1 | 1 | 1 | 1 | 1 |
| (Table 7) | | | | | | | | |
| 18. Sunflower oil | 2.5 | 2.5 | 4 | 4 | 2.5 | 2.5 | 2.5 | 2.5 |
| 19. Octyl pelargonate | 2.5 | 2.5 | 4 | 4 | 2.5 | 2.5 | 2.5 | 2.5 |

Notes to Table 8.
1. Trade Name for spray-dried, cold water-soluble maltodextrin reportedly having a DE range 4-7.
2. Trade Name for spray-dried, cold water-soluble maltodextrin reportedly having a DE range 9-12.
3. Trade Name for spray-dried, cold water-soluble maltodextrin reportedly having a DE range 13-17.
4. Trade Name for spray-dried, cold water-soluble maltodextrin reportedly having a DE range 16.5-19.5.

The compositions were prepared by separately blending together ingredient nos. 1-3 (Part 1) and ingredient nos. 5-16 (Part 2) until homogeneous. Part 1 was then added to Part 2 and mixed together until homogeneously blended to provide a visually uniform blue-colored granular powder portion (P). Ingredient no. 17, which was supplied as a pre-blended mixture, was then added slowly to the powder portion (P) under mixing agitation, followed by ingredient nos. 18 and 19, which were also supplied as a pre-blended mixture. The resulting granular mixture was maintained under stirring agitation until it was homogeneous and substantially free of discernible clumps. The clump-free granular mixture was then pulverized, using a powder grinder unit, to a substantively free-flowing Component (A) (i.e., generally left substantially no residue when passed through a mesh #30 sieve and a remainder in the range of about 3 to about 6% when passed through a mesh #50 sieve indicating an average particle size in the range of about 0.3 to about 0.5 mm).

For use as a conditioning hair lightener emulsion, one part by weight of Component (A) preferably is mixed with two parts by weight Component (B), and Component (B) is preferably an aqueous hydrogen peroxide containing composition.

The following three additional embodiment Examples 9 (I, J, and K) also were prepared similarly, except that the cationic polymer ingredient no. 7 was varied as described below. Composition Ex. (9-I) was prepared as described above by repeating the formulation of composition Ex. (9-H), except that ingredient no. 7 was omitted and directly replaced by 3 parts by weight polyquaternium-10 (INCI name for a polymeric quaternary ammonium salt of hydroxyethyl cellulose reacted with a trimethyl ammonium substituted epoxide, sold under the trade name UCARE® Polymer JR) having a high viscosity grade (UCARE® Polymer JR-30M). Composition Ex. (9-I) was less preferred for use in highly alkaline hair lightener emulsions having a pH>10 because polyquaternium-10 reportedly is subject to chemical hydrolysis in high pH environments.

Composition Ex. (9-J) was also prepared as described above by repeating the formulation of composition Ex. (9-H), except that ingredient no. 7 was omitted and directly replaced by 3 parts by weight of a cationic acrylamide copolymer powder (reportedly a non-crosslinked trimethylammonioethyl methacrylate chloride and acrylamide sold under the name POLYTEC™ 95). The conditioning hair lightener emulsion obtained by mixing one part by weight of Composition Ex. (9-J) with two parts by weight of aqueous Component (B), containing about 6% hydrogen peroxide, however, was judged difficult to spread through the hair.

Composition Ex. (9-K) was also prepared as described above by repeating the formulation of Ex. (9-J) except that the amount of cationic acrylamide copolymer powder was reduced to about 0.5 parts by weight and Polyquaternium-7 was added in an amount of about 2.5 parts by weight, and ingredient no. 13 was reduced to about 5 parts by weight and corn (Zea Mays) starch was added in amount of about 3 parts by weight. The conditioning hair lightener emulsion obtained by mixing one part by weight of Composition Ex. (9-K) with two parts by weight of aqueous Component (B), containing about 6% hydrogen peroxide was judged difficult to spread through the hair, however.

### EXAMPLE 10

Conditioning compositions for use as Component (A) of the hair lightener system are shown in Table 9 containing as hair protectant deswelling agent, a hydrogenated starch hydrolysate, and a total amount of about one weight percent of SFC, based on the weight of Component (A).

TABLE 9

| | |
|---|---|
| | Parts by Weight |
| Ingredient | (As Supplied) |

| (INCI/Common Name) | | (10-A) | (10-B) |
|---|---|---|---|
| 1. | Potassium persulfate | 29.4 | 29.4 |
| 2. | Sodium persulfate | 14.7 | 14.7 |
| 3. | Ammonium persulfate | 8.8 | 8.8 |
| 4. | Tetrasodium EDTA | 0.3 | 0.3 |
| 5. | Xanthan gum | 0.8 | 0.8 |
| 6. | Hydroxyethyl cellulose | 2.5 | 2.5 |
| 7. | Polyquaternium-6 (Note 1, Table 1) | 3 | 3 |
| 8. | STABILITE ™ SD30 (Note 1) | 19.4 | — |
| 9. | STABILITE ™ SD60 (Note 2) | — | 19.4 |
| 10. | Silica | 6 | 6 |
| 11. | Sodium metasilicate | 8.8 | 8.8 |
| 12. | Ultramarine Blue | 0.3 | 0.3 |
| 13. | (SFC) portion of Ex. 8E (Table 7) | 1 | 1 |
| 14. | Sunflower oil | 2.5 | 2.5 |
| 15. | Octyl pelargonate | 2.5 | 2.5 |

Notes to Table 9.
1. Trade Name for spray-dried, hydrogenated starch hydrolysate (polyglycitol) reportedly having an approximate polyols distribution of HP 1 (sorbitol) 2%; HP 2 (maltitol) 6%; HP 3 +92%; and a moisture content of not more than about 8%.
2. Trade Name for spray-dried, hydrogenated starch hydrolysate (polyglycitol) reportedly having an approximate polyols distribution of HP 1 (sorbitol) 1%; HP 2 (maltitol) 3.5%; HP 3 +95.5%; and a moisture content of not more than about 8%.

The compositions were prepared by separately blending together ingredient nos. 1-3 (Part 1) and ingredient nos. 4-12 (Part 2) until homogeneous. Part 1 was then added to Part 2 and mixed together until homogeneously blended to provide a visually uniform blue-colored granular powder portion(P). Liquid (SFC) portion ingredient no. 13, which was supplied as a pre-blended mixture, was then added slowly to the powder portion (P) under mixing agitation, followed by liquid ingredient nos. 14 and 15 (also supplied as a pre-blended mixture). The clump-free granular mixture was then pulverized, using a powder grinder unit, to a substantially free-flowing Component (A).

For use as a conditioning hair lightener emulsion, one part by weight of Component (A) is preferably mixed with two parts by weight Component (B), and Component (B) preferably is an aqueous hydrogen peroxide-containing composition.

### EXAMPLE 11

This example illustrates the beneficial conditioning effects on hair lightened with a conditioning hair lightener system of this invention, based on ease of combing in studies performed on tresses (Study 11-a and 11-b), and in salon studies.

Study 11-a.

Three tresses of Caucasian, natural medium brown-colored, virgin hair, (DeMeo Brothers) were prepared, each weighing about 4 grams and being about 18 cm in length.

The ease of combing of the tresses before and after lightening was evaluated using an automated Dia-Stron mini tensile tester equipped with Uvwin software. Each tress was combed before and after the lightening treatment and the amount of energy or work (joules) required to comb each tress was measured and recorded. The tresses were equilibrated at an ambient room temperature in the range of about 26 to about 27° C. in a chamber having a relative humidity of about 65% for a period of about 24 hours before each combing. The tresses were glued to a rectangular tab (sized about 3.8 by 5 cm) at the hair root portion, and the tabbed portion is secured to a tress anchor jaw unit of the tester and combed with a hard plastic comb downward from the root to end portion at a rate of about 120 millimeters (mm) per minute. Ten consecutive combing readings were taken for each tress. The highest force or peak load (grams-force) was used to measure the relative ease to detangle the hair during combing. A ratio of the reading before the lightening procedure relative to the reading after the lightening procedure was calculated to provide a combing index and used to assess the combing properties of the hair. A ratio value of less than 1 indicates easier combing.

The tresses were lightened as follows. A conditioning hair lightener emulsion (I) of this invention was prepared by mixing about 29.96 g of Composition Ex. (9-H) of Example 9 as Component (A) with about 56.7 g of a commercial aqueous Component (B), containing about 6% hydrogen peroxide and about 10% starch hydrolysate deswelling agent (AFFIRM® MoisturColor® 20 Volume, Moisturizing Creme Developer, Avlon Industries, Inc.) to provide a conditioning hair lightener emulsion (I) containing a total of about 0.4% by weight SFC based on the weight of emulsion (I). The three tresses were individually lightened by contacting each tress with about 32 grams of conditioning hair lightener emulsion (I) for a period of about 60 minutes. The conditioning hair lightener emulsion (I) was removed from each hair tress by rinsing with tepid tap water for about 3 minutes. The rinsed tresses were then each coated with a commercial cationic hair conditioner (AFFIRM® 5 in 1 Reconstructor, Avlon Industries, Inc.) for about 5 minutes, rinsed with tepid tap water for about 3 minutes, washed with a non-conditioning, normalizing-type shampoo having a pH in the range of about 4.5 to about 5.5, and then rinsed for about one minute with tepid tap water.

Study 11-b.

The procedure of Study 11-a was repeated, except that Component (A) was 29.96 g of composition Ex. (1-B2) of Example 1 containing SFC portion Ex. (1-D) and provided a conditioning hair lightener emulsion (II). Emulsion (II) had no cationic polymer, had deswelling agent only in Component (B) and about 0.4% by weight SFC on the weight of emulsion (II). The ease of wet and dry combing of the lightened tresses was also evaluated using the automated Dia-Stron mini tensile tester as described in Study 11-a.

The wet combing data for the tresses from Study 11-a showed a mean Wet Combing Peak Load Index (WCPLI) of about 0.61 and a Wet Combing Energy Index (WCEI) of about 0.62. The wet combing data for the tresses from Study 11-b showed a mean WCPLI of about 0.99 and a WCEI of about 1.1. Thus, the beneficial ameliorative effect of the conditioning hair lightener emulsion (I), Study 11-a was preferred because the wet combing property of the hair was judged substantially and significantly superior to that of conditioning hair lightener emulsion (II), Study 11-b.

The dry combing data for the tresses from Study 11-a showed a mean Dry Combing Peak Load Index (CPLI) of about 1.39 and a Dry Combing Energy Index (DCEI) of about 1.35. The dry combing data for the tresses from Study 11-b showed a mean DCPLI of about 1.2 and a DCEI of about 1.1. No statistical difference (p=>0.05) was found between the dry combing results obtained from either study.

Salon Studies. In a separate study, conditioning hair lightener emulsion (I) was prepared as described in Study 11-a, and the conditioning hair lightener emulsion (II) was prepared as described in Study 11-b, and compared to one another in “half-head” studies in a testing salon. Six female subjects, who had not previously bleached their hair, each received a hair lightening treatment with conditioning hair lightener emulsion (I) applied by professional beauticians to the hair on one side of her head and conditioning hair lightener emulsion (II) applied to the hair on the opposing side. The hair was contacted with each hair lightener emulsion for a total period of about 10 to about 20 minutes depending on the level of color lift described. Each hair lightener emulsion was removed by rinsing with tepid water, and the wet hair was subjectively evaluated for wet combing ease and softness. A commercial conditioner (AFFIRM® MoisturColor® for color treated hair, Avlon Industries, Inc.) was then applied to and distributed through the entire head of hair and left in contact therewith for a period of about 5 minutes, and removed by rinsing with tepid water, and the combing and softness of the hair again subjectively evaluated. The entire head of hair was then shampooed with a commercial conditioning shampoo (AFFIRM® MoisturColor® for color treated hair, Avlon Industries, Inc.) for about 3 minutes, the hair rinsed with tepid water, and wet combing evaluated. The hair was then blotted with a towel, blow dried with a hair dryer, and the dried hair was subjectively evaluated for dry combing ease, static flyaway, evidence of brittleness (i.e., raspy and dry).

The results showed that at all stages, when wet, the hair on the side lightened with the conditioning hair lightener emulsion (I), was discernibly easier to comb, and softer to the touch than the side lightened with conditioning hair lightener emulsion (II). When dry, the hair lightened with the conditioning hair lightener emulsion (I) was still judged slightly easier to comb than the side lightened with conditioning hair lightener emulsion (II). Otherwise the two lighteners were substantially similar in control of static flyaway, feel, color lifting (judged to be about 3 to 4 levels), and comfort (i.e., judged very comfortable with minimal irritation on the scalp). In the salon studies, the beauticians judged the Component (A) compositions as having an acceptable level of low dusting.

### EXAMPLE 12

This example illustrates the beneficial effect of a conditioning hair lightener emulsion of this invention containing deswelling agent in both Component (A) and Component (B), based on the Intermittent Stress Relaxation (ISR) Method previously described.

Study 12-a. A conditioning hair lightener emulsion (I) was prepared by mixing one part by weight of Composition Ex. (9-A) with two parts by weight of a commercial aqueous Component (B) containing about 6% hydrogen peroxide and about 10% starch hydrolysate (AFFIRM® MoisturColor® 20 Volume, Moisturizing Creme Developer, Avlon Industries, Inc.).

A tress was prepared of virgin, naturally brown Caucasian hair (DeMeo Brothers) about 1 g in weight and about 20 cm in length in which the hair fibers were mixed to provide a random blend of root-to-tip and tip-to-root orientation. About a 10 cm portion of the hair tress (referred to as the upper portion) was secured within a plastic tube to prevent contacting the upper portion of the hair tress with the hair lightener emulsion. The remaining exposed portion of the hair tress (referred to as the lower portion) was then contacted with a sufficient amount of conditioning hair lightener emulsion (I) to substantially uniformly coat the tress and lightened for about 60 minutes.

The conditioning hair lightener emulsion (I) was removed from the hair by rinsing the hair with tepid tap water for about 3 minutes. The rinsed hair was then coated with a commercial cationic hair conditioner (AFFIRM® 5 in 1 Reconstructor, Avlon Industries, Inc.) for about 5 minutes, followed by rinsing with tepid tap water for about 3 minutes, shampooing with a non-conditioning, normalizing-type shampoo having a pH in the range of about 4.5 to about 5.5 and then rinsed for about one minute with tepid tap water.

Eight fibers were randomly sampled from the hair tress and the strength of the hair evaluated by measuring the ISR of the untreated portion and of the lightened portion of the same fiber and then calculating the ISR Index in this manner for each of the fibers.

Study 12-b. The procedure of study 12-a was repeated except that the hair lightener emulsion was prepared with one part by weight of Composition Ex. (9-B) as Component (A) to provide a hair lightener emulsion (II).

Study 12-c. The procedure of study 12-a was repeated except that the hair lightener emulsion was prepared with one part by weight of Composition Ex. (9-C) as Component (A) to provide a hair lightener emulsion (III).

Study 12-d. The procedure of study 12-a was repeated except that the hair lightener emulsion was prepared with one part by weight of Composition Ex. (9-D) as Component (A) to provide a hair lightener emulsion (IV).

Study 12-e. The procedure of study 12-a was repeated except that the hair lightener emulsion I was prepared with one part by weight of Composition Ex. (9-E) as Component (A) to provide a hair lightener emulsion (V).

Study 12-f. The procedure of study 12-a was repeated except that the hair lightener emulsion I was prepared with one part by weight of Composition Ex. (9-F) as Component (A) to provide a hair lightener emulsion (VI).

Study 12-g. The procedure of Study 12-a was repeated except that the hair lightener emulsion I was prepared with one part by weight of Composition Ex. (9-G) as Component (A) to provide a hair lightener emulsion (VII).

Study 12-h. The procedure of Study 12-a was repeated except that the hair lightener emulsion I was prepared with one part by weight of Composition Ex. (9-H) as Component (A) to provide a hair lightener emulsion (VII).

Study 12-i. The procedure of Study 12-a was repeated except that the hair lightener emulsion (I) was prepared with one part of Composition Ex. (10-A) as Component (A) to provide a hair lightener emulsion (IX).

Study 12-j. The procedure of Study 12-a was repeated except that the hair lightener emulsion (I) was prepared with one part of Composition Ex. (10-B) as Component (A) to provide a hair lightener emulsion (X).

Based on the weight of the emulsion (I-X) the conditioning hair lightener emulsions in studies 12 (a-j) contained about 0.3% to about 0.35% by weight SFC.

Relative to untreated control hair (ISR Index=1) the average ISR Index for the lightened fibers are shown in Table 10.

TABLE 10

| Emulsion | Average ISR Index |
|---|---|
| I, Ex. 12-a | 0.75 |
| II, Ex. 12-b | 0.86 |
| III, Ex. 12-c | 0.8 |
| IV, Ex. 12-d | 0.87 |
| V, Ex. 12-e | 0.84 |
| VI, Ex. 12-f | 0.82 |
| VII, Ex. 12-g | 0.87 |
| VIII, Ex. 12-h | 0.87 |
| IX, Ex. 12-i | 0.73 |
| X, Ex. 12-j | 0.82 |
| Control (Untreated) | 1 |

The data show that each of the conditioning hair lightener emulsions (II) through (VIII), and (X) ameliorated the loss in strength (i.e., elasticity) of the fibers to a decrease of not more than about 20% (e.g., in a range of about 13 to about 18%), and conditioning hair lightener emulsions (I) and (IX) ameliorated the loss in strength (elasticity) of the fibers to a decrease of not more than about 27% (e.g., in a range of about 25 to about 27%). Based on a statistical analysis of the ISR data (by Tukey HSD technique which uses a Harmonic Mean Sample Size of 80.000), there was no statistical difference (p=>0.05) between the ISR indices of tresses lightened with conditioning hair lightener emulsions (II), (IV), (VII), and (VIII), so the amount of deswelling agent present in conditioning hair lightener emulsion (VIII) was judged optimized.

### EXAMPLE 13

This comparative example illustrates the beneficial effect of a conditioning hair lightener emulsion of this invention containing both SFC and deswelling agent in Component (A), based on the Intermittent Stress Relaxation (ISR) Method.

A conditioning hair lightener emulsion (I) was prepared by mixing one part by weight of Composition Ex. (9-H) (Table 8) with two parts by weight of a commercial aqueous Component (B) containing about 6% hydrogen peroxide and about 10% starch hydrolysate (AFFIRM® MoisturColor® 20 Volume, Moisturizing Creme Developer, Avlon Industries, Inc.).

For comparison, a hair lightener composition (CC) containing no SPC or deswelling agent in Component (A) was prepared by following the procedure described in Example 9 for Composition Ex. (9-H) (Table 8), except that ingredient nos. 12 and 17 were omitted and directly replaced by (i.e., total of 11 parts by weight) corn (Zea Mays) starch. A comparative hair lightener emulsion (II) was then prepared by mixing one part by weight of composition (CC) with two parts by weight of the commercial aqueous Component (B) containing about 6% hydrogen peroxide and about 10% starch hydrolysate (AFFIRM® MoisturColor® 20 Volume, Moisturizing Creme Developer, Avlon Industries, Inc.).

The average intermittent stress strain relaxation (ISR) value for individual hair fibers was determined in a cycle of constant strain of about 0.5% of the length of the fiber for a period of about 30 seconds followed by a relaxation of the strain for about 30 seconds for a total of 10 cycles to provide pre-tested fibers before the hair lightening procedure. The pretested fibers were embedded at the root portion in a tress prepared of virgin, naturally brown Caucasian hair (DeMeo Brothers) about 2 g in weight and about 18-20 cm in length (8 pre-tested fibers per tress). The tress containing the embedded pre-tested fibers was then subjected to the following hair lightening procedure. About 8 g of hair lightener emulsion (I) was applied to the tress (to provide a bath:hair weight ratio of about 4:1), the tress was covered with a plastic wrap, and lightened for a period of about 60 minutes at ambient room temperature. The lightened tress was uncovered and the hair lightener emulsion (I) was removed from the tress by rinsing with tepid tap water for about 3 minutes. The rinsed tress was then coated with a commercial cationic hair conditioner (AFFIRM® 5 in 1 Reconstructor, Avion Industries, Inc.) for about 5 minutes, followed by rinsing with tepid tap water for about 3 minutes, shampooing with a non-conditioning, normalizing-type shampoo having a pH in the range of about 4.5 to about 5.5 and then rinsing for about one minute with tepid tap water. The now lightened, 8 pre-tested fibers were removed from the hair tress and the ISR value again determined as described above and the average ISR Index was calculated.

For comparison, the foregoing procedure was repeated, except that the comparative hair lightener emulsion (II) was employed.

The average ISR Index obtained with the conditioning hair lightener emulsion (I) had an average ISR Index of 0.83, which was judged statistically different (p=<0.05) from the average ISR Index of 0.77 obtained with emulsion (II) prepared with the comparative hair lightener (CC). Thus, the hair lightened with SFC and deswelling agent present in Component (A) (i.e., Composition Ex. 9-H) was judged significantly stronger than the hair lightened without SFC or deswelling agent present in Component (A) (i.e., comparative hair lightener (CC)).

### EXAMPLE 14

This example illustrates the beneficial amelioration in hair porosity by using a conditioning hair lightener system of this invention, based on Liquid Retention (porosity) procedure, previously described.

The conditioning hair lightener emulsion (VIII) of Study 12-h of Example containing deswelling agent in Component (A), was prepared as described in Study 12-h and a comparative hair lightener emulsion containing no deswelling agent or SFC in Component (A) was prepared. The comparative Component (A) was prepared by mixing one part of the powder portion (P) of composition Ex. (1-B4) containing no (SFC) portion (and no deswelling agent) with two parts by weight of a commercial aqueous Component (B) containing about 6% hydrogen peroxide and about 10% starch hydrolysate (AFFIRM® MoisturColor® 20 Volume, Moisturizing Creme Developer, Avlon Industries, Inc.).

Two tresses of virgin, medium brown color, Caucasian hair (De Meo Brothers), each weighing about 4 grams, and about 18 cm in length, were prepared. One of the tresses was lightened by contacting it with about 32 g conditioning hair lightener emulsion (VIII) of Ex. 12-h for about 30 minutes. The second tress was similarly treated, except that the comparative hair lightener emulsion was used. In both studies, the hair lightener emulsions were removed from the hair by rinsing the hair with tepid tap water for about 3 minutes. The rinsed tresses were then coated with a commercial cationic hair conditioner (AFFIRM® 5 in 1 Reconstructor, Avlon Industries, Inc.) for about 5 minutes, rinsed with tepid tap water for about 3 minutes, washed with a non-conditioning, normalizing-type shampoo having a pH in the range of about 4.5 to about 5.5 and then rinsed for about one minute with tepid tap water.

The lightened tresses were allowed to air dry at ambient room temperature in the range of about 26 to about 27° C., and a relative humidity of about 65% for at least about 12 hours. About eight samples, each weighing about 500 milligrams, were removed from each lightened tress, placed in separate uncapped weighing bottles, and equilibrated at ambient room temperature and relative humidity of about 65% for about 24 hours. The equilibrated lightened hair samples were then weighed using a microbalance (Mettler-Toledo) and the weight of each hair sample was recorded to obtain the initial weight (W) of the equilibrated lightened hair.

Each weighed equilibrated lightened hair sample was then separately immersed in a glass vessel containing about 60 ml of deionized water and soaked for a period of about 30 minutes. Each soaked hair sample was then removed from the water using stainless steel forceps and transferred to a centrifuge tube containing a wire-gauze at the bottom of the tube to prevent the water-swollen hair from coming in contact with draining water. The centrifuge tube was sealed and the water-swollen hair was centrifuged at a speed of about 3500 revolutions per minute for about 10 minutes using a laboratory centrifuge (Drucker Model 708E/EL). After centrifuging, each hair sample was removed from the centrifuge tube and weighed again in the microbalance to obtain the weight of the water-swollen lightened hair (Wa) after centrifuging. The porosity, based on % liquid retained, was then calculated by the formula:

Liquid retention (porosity)=([Wa−(W−0.162W)]/Wa)×100.

The results were compared against an untreated control tress (not lightened).

The data showed that, on average, the control (untreated) hair had a liquid retention of about 27.8%, the hair lightened with the conditioning hair lightener emulsion (VIII) of Ex. 12-h had a liquid retention of about 29.3%, and the hair lightened with the comparative hair lightener emulsion had a liquid retention of about 31.3%.

The foregoing examples show that a conditioning hair lightener of this invention prepared with Component (A) containing a self-emulsifying, fatty-acid derived conditioner (SFC), and preferably a hair protectant deswelling agent, ameliorates one or more deleterious effect associated with lightening the color of hair with peroxy salt-containing hair lighteners under relatively high alkaline conditions.

The foregoing examples are intended to be illustrative and not limiting. Numerous variations and modifications may be effected without departing from the true spirit and scope of the invention.

1. A substantially anhydrous, free-flowing composition suitable as a component for preparing a conditioning hair lightener emulsion comprising:
an effective hair conditioning amount of a water-dispersible, self-emulsifying, fatty acid-derived conditioner;
an effective hair lightening amount of at least one peroxy salt compound;
optionally, an effective hair protective amount of a hair protectant, deswelling agent; and
optionally, a water-soluble or water-dispersible cosmetic adjuvant;
wherein the composition is maintained as a substantially anhydrous, substantially free-flowing component until being mixed with a separately prepared component comprising an aqueous medium, and wherein, upon being so mixed, substantially immediately forms a conditioning hair lightener emulsion.

2. The composition of claim 1 wherein the self-emulsifying, fatty acid-derived conditioner comprises a fatty ester, an ethoxylated glyceride, a fatty alcohol, a fatty ether, and any combination thereof, and any formulation thereof optionally containing at least one hydrophilic surfactant.

3. The composition of claim 1 wherein the self-emulsifying, fatty acid-derived conditioner comprises at least one polyhydric ester selected from the group consisting of a C₃-C₄ polyol ester of a C₆-C₂₂ fatty acid; a glyceryl ester of a C₆-C₂₂ fatty acid and at least one acid selected from the group consisting of citric acid, lactic acid and succinic acid; and a polyethoxylated C₁₂-C₁₈ acylated sorbitol ester.

4. The composition of claim 1 wherein the self-emulsifying, fatty acid-derived conditioner comprises predominantly a C₈-C₁₀ fatty acid ester of a polyol selected from the group consisting of glycerin, propylene glycol, butylene glycol and mixtures thereof.

5-7. (canceled)

8. The composition of claim 1 wherein the self-emulsifying, fatty acid-derived conditioner comprises a caprylic/capric triglyceride, a glyceryl cocoate/citrate/lactate, a PEG-40 sorbitan peroleate or a combination of two or more of the foregoing.

9. The composition of claim 1 wherein the peroxy salt is selected from the group consisting of an alkali metal persulfate, ammonium persulfate, and mixtures thereof.

10. The composition of claim 1 wherein the composition comprises a hair protectant, deswelling agent selected from the group consisting of a polyol and a cationic polymer.

11. The composition of claim 10 wherein the polyol is a carbohydrate.

12. The composition of claim 11 wherein the carbohydrate is a starch hydrolysate.

13. The composition of claim 12 wherein the starch hydrolysate is a maltodextrin.

14. The composition of claim 1 containing a cosmetic adjuvant comprising a cationic polymer as an auxiliary hair conditioning agent.

15. The composition of claim 14 wherein the cationic polymer is polyquaternium-6.

16. A conditioning hair lightener emulsion prepared from at least two separate components, (A) and (B), wherein Component (A) is a substantially anhydrous, free-flowing composition of claim 1 and Component (B) comprises an aqueous medium containing an effective hair lightening amount of hydrogen peroxide or source thereof, and wherein the hair lightener emulsion has a pH of at least about 8.

17. The conditioning hair lightener emulsion of claim 16 wherein a hair protectant, deswelling agent is present in at least one of Component (A) and (B).

18. A method of lightening hair comprising the steps of:
(I) contacting substantially dry hair with the conditioning hair lightener emulsion of claim 16 and distributing the composition therethrough,
(ii) maintaining the applied conditioning hair lightener emulsion in contact with the hair for a period sufficient to visibly lighten the color of the hair to a desired shade level, to provide lightened hair, optionally contacting the so-lightened hair with an aqueous acidic medium having a pH of not more than about 5, and
(iii) removing the hair lightener emulsion from the lightened hair.

19. The method of claim 18 wherein step (iii) is performed by rinsing the hair with water.

20. (canceled)

21. The method of claim 18 wherein the post-lightening aqueous acidic medium, when employed, contains a cationic polymer.

22. The method of claim 18 wherein the lightened hair is contacted with a post-lightening cationic conditioner after step (iii) and the post-lightening cationic conditioner is removed from the conditioned, lightened hair with water.

23. The method of claim 18 further including the step (iv) of washing the lightened hair with a shampoo having a pH in the range of about 4 to about 6.

24. A conditioning hair lightener system comprising at least two components, Component (A) and Component (B), wherein:
Component (A) is a composition of claim 1 and
Component (B) is an aqueous medium containing hydrogen peroxide or hydrogen peroxide source,
wherein Component (A) and Component (B) are maintained separate, and substantially immediately before use, Component (A) and Component (B) are mixed together to provide a conditioning hair lightening emulsion having a pH of at least about 8.

25. The conditioning hair lightener system of claim 24 further including a separate component comprising a post-lightening aqueous acidic medium having a pH of not more than about 5; a post-lightening cationic hair conditioner; a post-lightening shampoo having a pH in the range of about pH 4 to about 6; or combination of two or more of the foregoing.

26-27. (canceled)

28. The conditioning hair lightener system of claim 24 wherein Component (A) includes a cationic polymer.

29. The conditioning hair lightener system of claim 24 wherein at least one of Component (A) or Component (B) includes a hair protective, deswelling agent.

30. The conditioning hair lightener system of claim 25 wherein the post-lightening, aqueous acidic medium, when present, includes a nonionic polymer, a cationic polymer or combination thereof.

31. An article of manufacture comprising a kit containing at least one composition of claim 1 in packaged form.

32. (canceled)

33. The article of manufacture of claim 31 further including at least one separately packaged component selected from the group consisting of:
an aqueous medium containing hydrogen peroxide or hydrogen peroxide source;
a post-lightening aqueous acidic medium having a pH of not more than about 5;
a post-lightening cationic hair conditioner;
a post-lightening shampoo having a pH in the range of about 4 to about 6;
a hair lightening implement; and
instructional indicia.

34. (canceled)
